(19) United States
(12) Patent Application Publication
(10) Pub. No.: US 2019/0184391 A1
(43) Pub. Date: Jun. 20, 2019

(54) FILTERING SYSTEMS UTILIZING NANO NEEDLES TO PIERCE CELL WALLS IN A FLUID FLOW THROUGH MICRO OR NANO STRUCTURES

(71) Applicant: Cargico Microfluidics Corporation, Campbell, CA (US)

(72) Inventor: Yen Kuen Shiau, Hacienda Height, CA (US)

(21) Appl. No.: 16/220,674

(22) Filed: Dec. 14, 2018

Related U.S. Application Data
(60) Provisional application No. 62/708,618, filed on Dec. 15, 2017.

(51) Int. Cl. B01L 3/00; A01N 33/12; B01D 35/30; B01D 37/02; G01N 1/28

(52) U.S. Cl. CPC B01L 3/502; A01N 33/12; B01D 35/30; B01D 37/025; G01N 2333/195; A61L 2300/406; B01D 2257/91; B01L 2300/0672; B01L 2300/0896; G01N 1/286

(57) ABSTRACT

Described herein is an antibacterial filtering system. The system includes at least one microstructure disk contained in a housing through which there is a fluid flow containing the cells to be eliminated. The microstructure disk includes a plurality of raised microstructures. The microstructures are positioned to form inlet channels and outlet channels for the fluid flow to pass through. The microstructures are coated with an antimicrobial material that is bonded to the microstructures. The antimicrobial material includes at least one quarternary ammonium salt (QAS) and/or at least one siliylated polyvinylpyrrolidone (PVP) quarternized salt.

### CROSS REFERENCE TO RELATED APPLICATIONS

This application claims the priority benefit of U.S. Provisional Application No. 62/708,618, filed on Dec. 15, 2017, which is hereby incorporated herein by reference in its entirety, including all references and appendices cited therein, for all purposes.

### FIELD OF THE INVENTION

This disclosure relates to piercing cell walls utilizing a fluidic system with channels that are formed by micro and/or nano meter scale structures deployed on the surface of a substrate. The structures are positioned to create channels to control the flow of fluids through a device embodying the system. The micro and/or nano meter scale structures are coated with a material that forms positively charged nano needles. The positively charged nano needles attract the naturally negatively charged cells. The cell walls are pierced on contact with the nano needles, thereby killing the cells and releasing the cell contents into the carrier fluid.

### SUMMARY OF THE INVENTION

In various embodiments of the present invention, a fluidic system includes, micro and/or nanometer scale structures positioned on the surface of a substrate to form channels. The structures are positioned to create micro and nano channels to control the flow of fluids to and from the system channels.

Some or all of the microstructure surfaces are coated with positively charged nano needles. These charged nano needles attract and pierce negatively charged cell walls. The piercing of cell walls is useful to kill a particular type of cell such as harmful bacteria or the needles can be used to open a cell to access the internal components for analysis. A wide range of materials can be used to fabricate the systems, from inexpensive plastics to materials that are durable at high temperatures such as silicon. The nano needle coating process requires one of a specific type of material. With many substrate materials an intermediate coating that provides the required material for nano needle coating might be required.

A number of embodiments of the present disclosure are directed to a device for the destruction of cell walls. For discussion a simple enclosure assembly is disclosed to discuss how the system elements could be deployed. The invention is not limited to usage in the disclosed enclosure assembly. One skilled in the art of enclosure assembly design could develop many other ways to deploy the system elements. Generally, a fluid enters the system element from the edge of a disk or panel. On the surface of the system element there are primary and secondary structures to create micro or nano channels. The primary structures create channels that direct the fluid to the secondary structures. All or part of the fluidic micro or nano structures are coated with nano scale needles that have a positive electrical charge. This positive charge attracts and the sharp end of the nano needle punctures and destroys the cell wall. This in effect kills the cell and releases the internal components of the cell into the fluid.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 is a sectioned perspective view of a filtering system according to the present disclosure.

FIG. 2 is a perspective view of the microstructure filtering disk assembly shown in FIG. 1.

FIG. 3 is a perspective view showing a single microstructure filtering disk.

FIG. 4 is a closeup view of a section of the disk shown in FIG. 3 showing the micro or nano-scale structures.

FIG. 5 is another perspective closeup view of a pair of filtering disks mated to each other.

FIG. 6 is a detail sectional view of a pair of filtering disks.

FIG. 7 is a perspective view of an alternate embodiment of the invention with a partial break away section.

FIG. 8 is a perspective view of a single panel from the assembly illustrated in FIG. 7.

FIG. 9 is a closeup view of the panel shown in FIG. 8 depicting one embodiment of the micro or nano structures.

FIG. 10 is a top view of the panel shown in FIG. 8.

FIG. 11 shows the chemical diagram of an exemplary quaternary ammonium salt (QAS) utilized in the present invention.

FIG. 12 shows the chemical diagram of an exemplary silylated PVP quantized salt.

FIG. 13a shows a diagram of the electrical charge and major components of a cell with the cell wall intact.

FIG. 13b shows the electrically charged nano needles.

FIG. 13c illustrates the attraction of the negatively charged cell to the positively charges nano needles, resulting in the piercing of the cell wall.

FIG. 14 shows the flow of a fluid with cells through a channel formed from micro or nano structures.

FIG. 15 shows an alternate embodiment of channels formed from micro or nano structures.

### DETAILED DESCRIPTION OF EXEMPLARY EMBODIMENTS

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

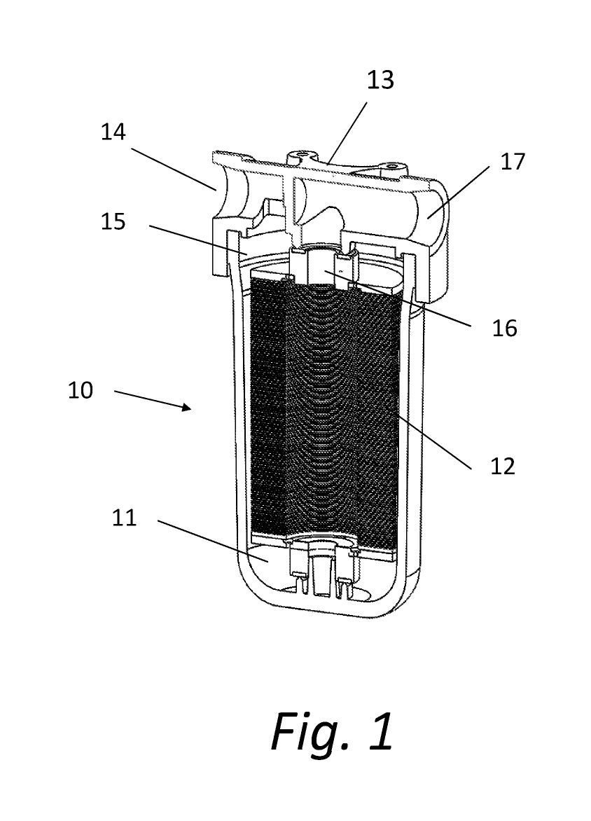

The present disclosure is generally directed to filtering systems that function by destroying cell walls of bacteria or other organisms in a fluid flow. Referring first to FIG. 1, a filtering system 10 utilizes a housing 11 that contains micro or nano structure disks 12 on which fluidic channels are created with micro and nano meter scale structures formed on the surface of a substrate. In light of the present disclosure, it will be readily apparent to those skilled in the art that the scale employed in a given implementation will be dependent on the parameters of that given implementation. Accordingly, for ease of drafting, the term “micro” will be used throughout this disclosure to refer to the sizes employed. It is understood that the structures described may be in the nano realm, or conversely in larger parameters if required for a given embodiment.

The housing 11 includes a main body and a fitting housing 13 that enables the device to be secured in position. The device receives a fluid flow at an inlet 14. The fluid that acts as a carrier in the device may be a gas or a liquid depending on the purpose of the particular system 10. The fluid flow flows from the inlet 14 to an inlet plenum 15, where it is directed to the microstructure disks 12. As the fluid flows across the disks 12, the fluid flow contacts raised microstructures on the surface of the microstructure disks 12. The microstructures and the composition of the fluid flow are discussed in further detail below. After the fluid flow passes across the surface of the microstructure disks 12, the fluid flows into an outlet plenum 7. The fluid then exits the system housing 11 via an outlet 17.

The system embodiment shown in FIG. 1 illustrates a large plurality of microstructure disks 12. The number of disks 12 is of course dependent upon the flow requirements of the system, the size of the disks utilized, and other factors established by the purpose and use of the system. Many fewer microstructure disks 12, perhaps only a single disk 12, may be required and deployed in a given system.

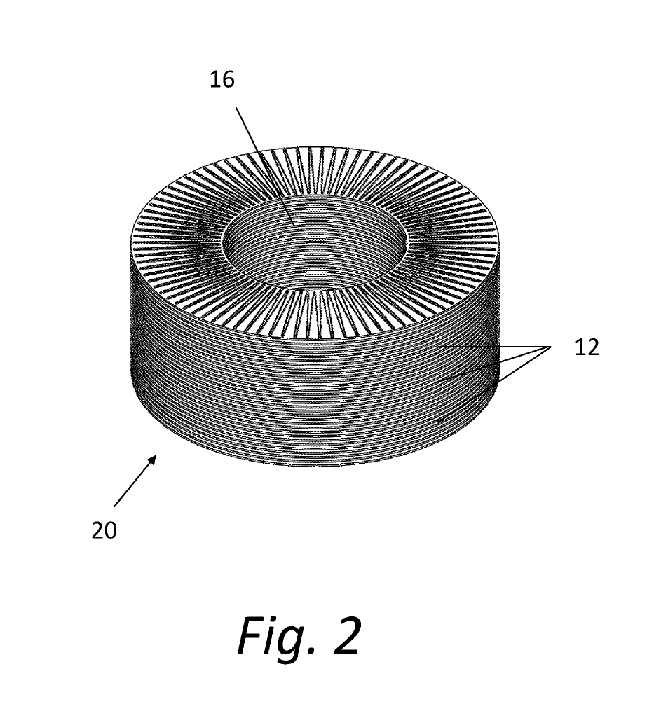

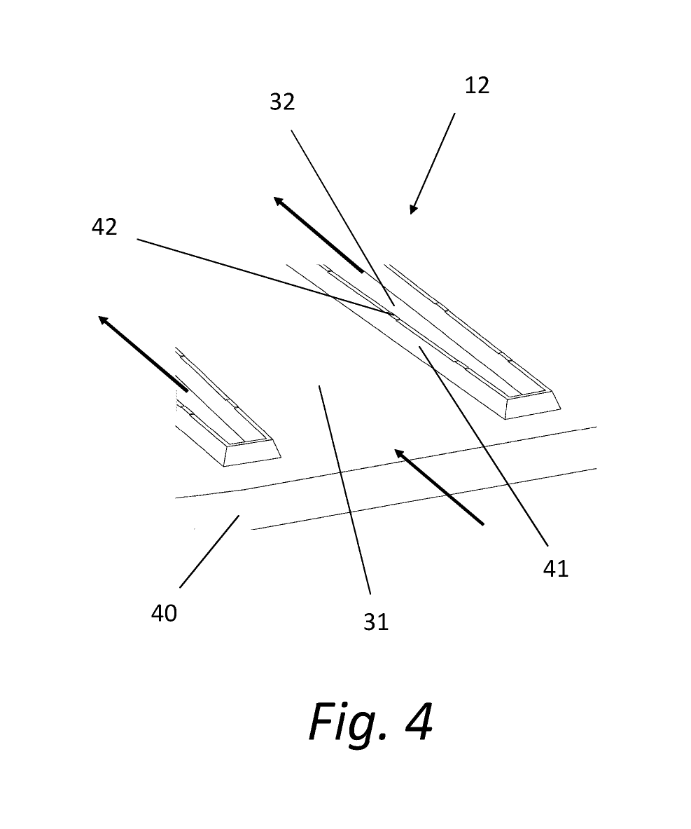

FIG. 2 illustrates in more detail an exemplary disk assembly 20 like that shown in FIG. 1. The disk assembly 20 includes one or multiple microstructure disks 12. In the configuration illustrated in FIG. 2, the disk assembly 20 includes several hundred individual disks 12. The disks 12 are typically stacked on top of one another, although other configurations may be utilized. A top surface of a first microstructure disk 12 is generally in physical contact with a bottom surface of an adjacent disk 12. The disks 12 may be secured in their positions relative to each other with physical features as most easily seen in FIG. 4. The microstructure disks 12 are typically made from thin plastic material, although many other materials with suitable characteristics will suffice.

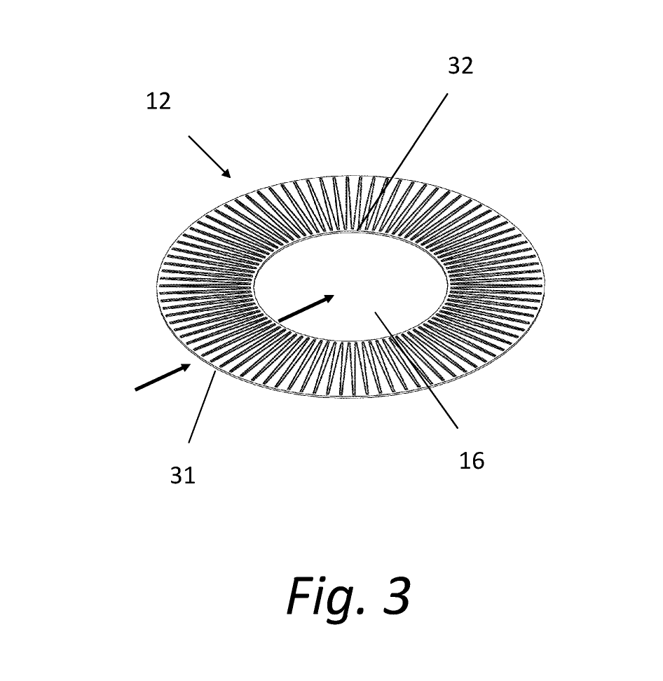

FIG. 3 shows an individual microstructure disk 12. As the carrier fluid flows into the filtering system 10, the fluid flows through the inlet plenum 15 where the fluid is received into inlet channels 31 that are formed on the surface of the disk 12. The inlet channels 31 are typically tapered inwards towards the center of the disk 12. A radial array of inlet channels 10 is created around the surface of the microstructure disk 12. The fluid flows across the face of the microstructure disk 12, through outlet channels 32, and into the outlet plenum 16. The inlet channels 31 and the outlet channels 32 are typically V shaped, although many other shapes could be used if desired.

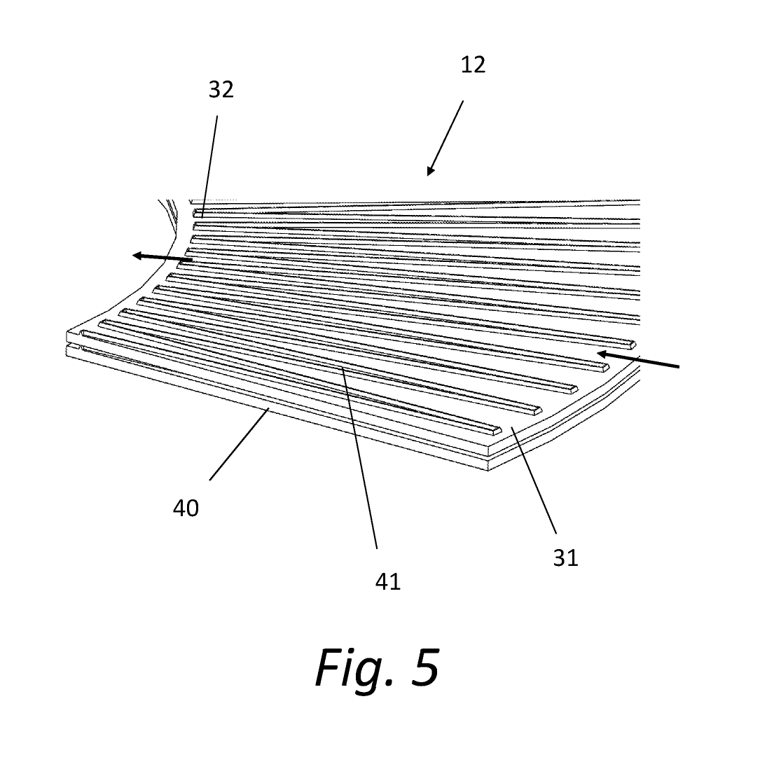

FIGS. 4 and 5 depict exemplary surface features—microstructures 41—that are formed on the surface of the substrate 40 of the microstructure disk 12. The microstructures 41 define the walls of the inlet 31 and outlet 32 channels. The microstructure 41 channel walls are typically in the range of 50 to 200 microns tall. The actual height would of course depend on manufacturing constraints and flow requirements.

Located atop the microstructures 41 are spacers 42. The spacers 42 extend only a short distance along the microstructures 41. The spacers 42 are typically spaced approximately 10× that of their length along the microstructures 41. When a lower surface of an adjacent disk 12 with depressions that mirror the spacing and depth of the microstructures 41 in the subject disk 12, the inlet 31 and outlet 32 channels are defined. An alternative structure would be to provide a flat piece of material without structures on the topside, as would be the case for the terminal disk 12. For most applications the height of the spacers 42 on the microstructures 41, and therefore the corresponding height of the micro channels 31, 32, is from a few microns to possibly a few decades of nanometers. It should be noted that the scale of the height of the microstructures 41 (nanometers in height) and the scale of the spacers 42 (many microns in height) are not the same. The spacers 42 would not be visible to the eye in relation to the microstructures 41 if drawn to scale.

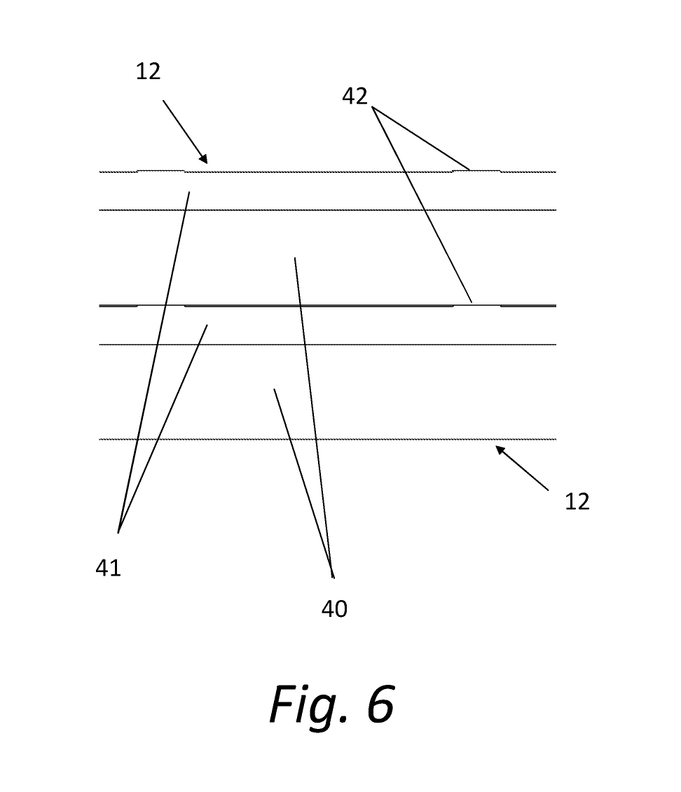

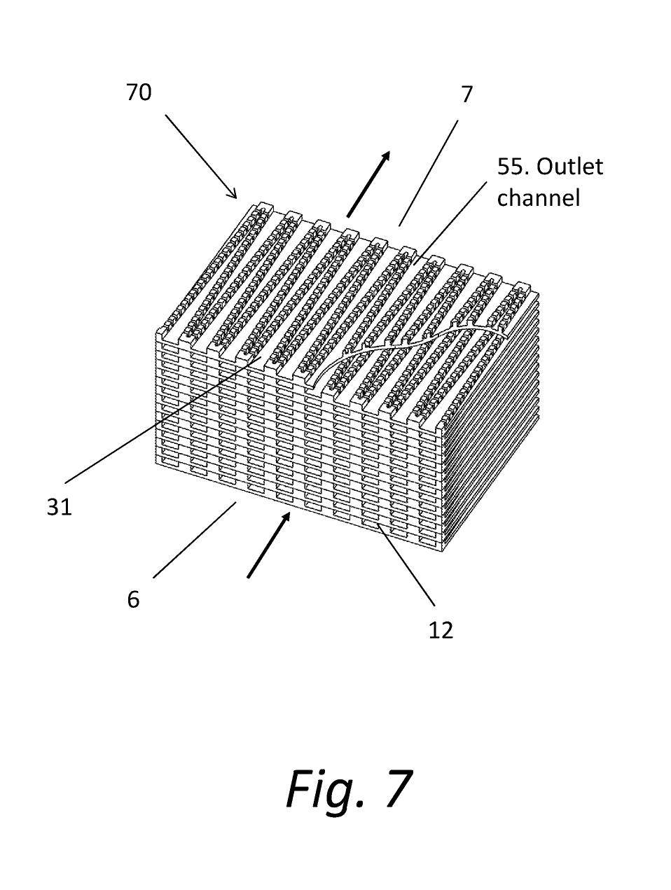

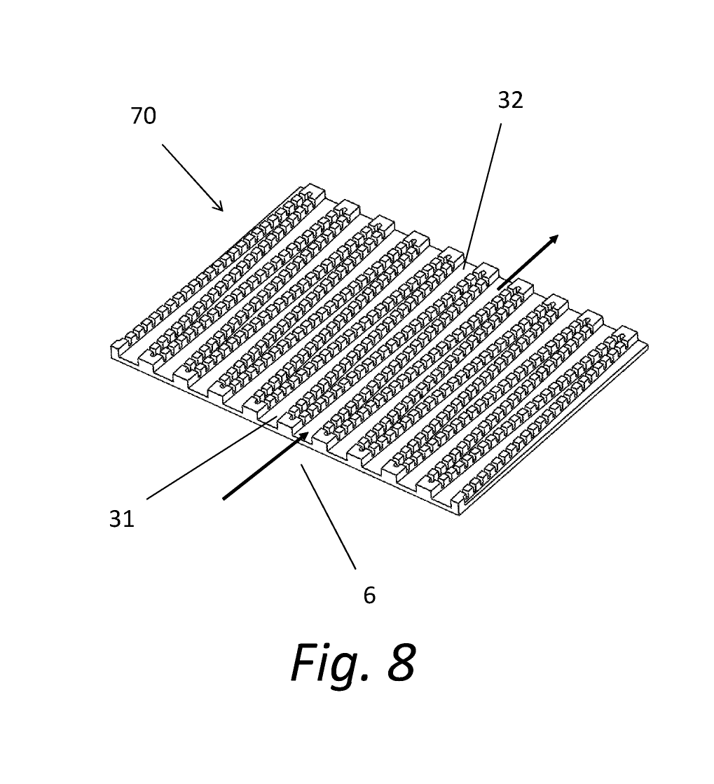

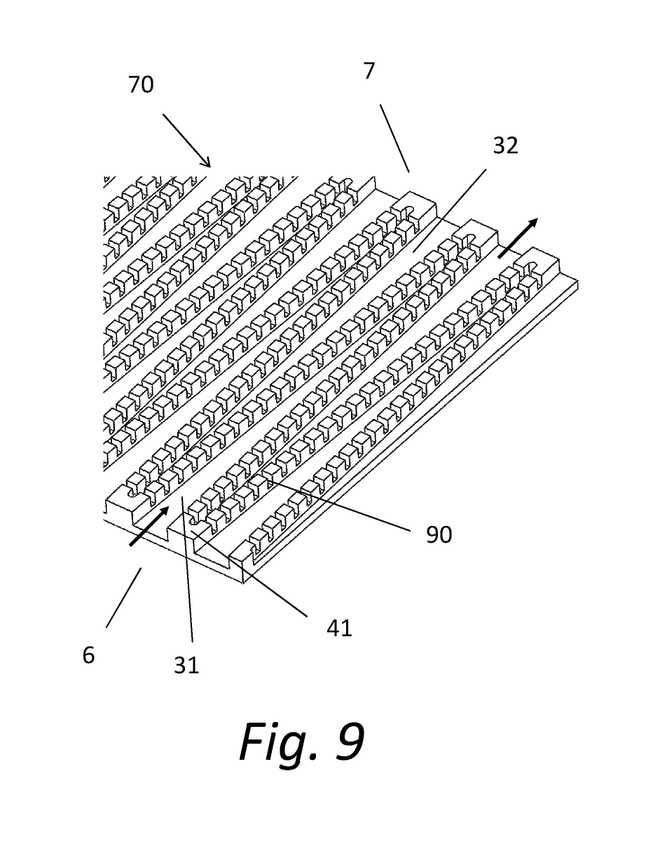

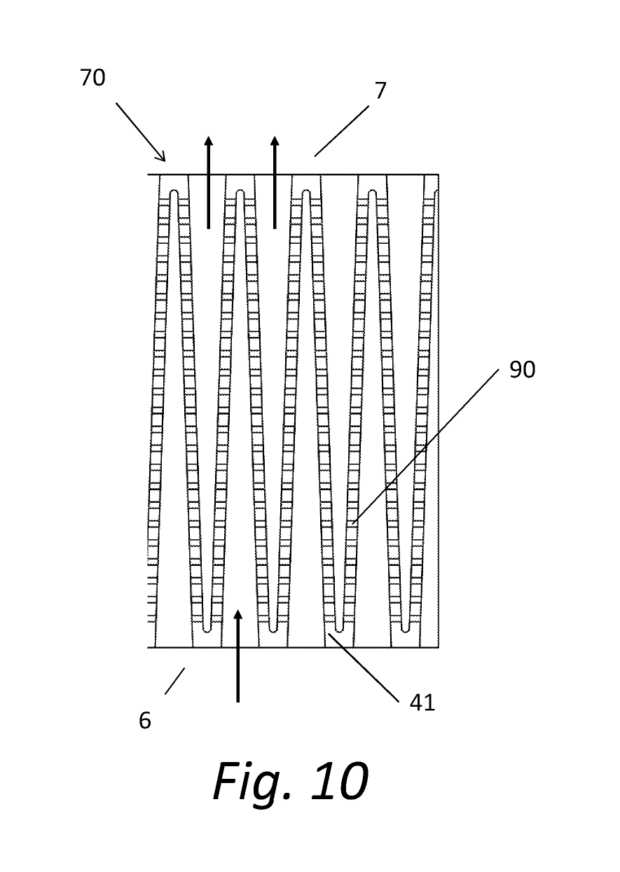

FIG. 6 is a detailed cross section showing two microstructure disks 12 stacked on top of each other. FIG. 7 illustrates an exemplary disk array 70 that includes a plurality of microstructure disks 12. It is envisioned that most filter systems according to the present invention will include a disk array 70. The disk array 70 can be of various configurations, sizes, and with varying sizes of channels therein. FIG. 8 illustrates an exemplary configuration for the microstructures 41 formed on the substrate 40 of the microstructure disk 12. FIGS. 9 and 10 show more detailed views of the raised microstructures 41. In FIGS. 9 and 10, it can be seen that in embodiments utilizing this or a similar construction for the microstructures 41, cross channels 90 that allow cross flow between the main inlet 31 and outlet channels 32 are formed between the segments of the microstructures 41.

The height of the spacers 42 and the corresponding size of the micro/nano channels 31, 32 creates a mechanical barrier that restricts the passage through the filtering system of cells larger than the size of the cross section of the channels 31, 32. Rigid cells larger than the cross section would not pass through the micro/nano channels 31, 32. Cells larger than the cross section of the channels 31, 32 that have flexible cell walls might deform sufficiently so that the cell could pass through. Flow rates and pressures affect to what degree such oversize cells would pass through the micro/nano channels 31, 32.

In an exemplary embodiment, the height of a microstructure disk 12 might be only approximately 200 microns. A typical disk assembly 20 might contain a few hundred microstructure disks 12. Assuming an embodiment utilizing 300 microstructure disks 12 with a thickness of 200 microns, the disk assembly 20 would be only about 60 mm (2.36″) tall.

Dimensions of the filtering system are determined by the requirements of a given installation. Because the cells being treated must be able to flow through the disk assembly 20, the dimensions of the system are a function of the size of the cells to be treated. To accommodate the fluid flow required in a given system, the inlet and outlet channels 31, 32, must be large enough to accommodate a reasonable flow rate. Typically this would mean that the channels 31, 32 would have a height of approximately 50 microns or greater, but generally no more than 300 microns. The size of bacteria can range from 100 nm to 1.5 um, but the majority of bacteria fall between 200 nm to 1000 nm. The size of the nano needles and the system in general is a function of the bacteria that is desired to be eliminated.

In order to destroy cells (typically bacteria) in the fluid flow, the microstructures 41 on the disks 12 in a filtering system disk assembly 20 are treated so that nano needles are formed on the surface of the microstructures 41. The process used to create nano needles requires that materials of specific groups be applied to the subject surface. The microstructure disk 12 substrate may be fabricated from a nano needle formation appropriate material. Alternatively, the disk 12 and the microstructures 41 can be formed from another material, and then coated with a thin film of nano needle formation appropriate material. Proper foundation materials (substrates) for nano needle creation include SiO₂, Al₂O₃, ALN, ZrO₂, CeO₂, TiO₂, SiC, ZnO₂, Si₃N₄, ITO and other similar materials sharing the requisite properties. If substrates are to be coated, processes such as sputtering and physical vapor deposition may be utilized.

The general process of creating nano needles on a substrate is known in the art. Specific examples of patents teaching the coating of a substrate with nano needle forming materials include U.S. Pat. No. 6,251,417, issued Jun. 26, 2001; U.S. Pat. No. 6,715,618, issued Apr. 6, 2004; U.S. Pat. No. 6,780,332, issued Aug. 24, 2004; U.S. Pat. No. 7,468,098, issued Dec. 23, 2008; and U.S. application Ser. No. 13/541,471, filed Jul. 3, 2012, since abandoned. Each of these patents and applications is hereby incorporated by reference herein in its entirety for all purposes.

The process of creating the nano needles on the proper surface is generally initiated by coating a preformed disk with an antimicrobial coating material. The antimicrobial coating material reacts with the SiO₂ (or other alternative materials) on the surface of the substrate 40 to form a strong covalent bond or bonds via the presence of Van der Waals forces. The coating process may be any of immersion, dipping, spraying, aerosolizing, nebulizing, brushing, curtain coating, roller painting, silk screening, wash coating, lithography, ink jetting, and the like.

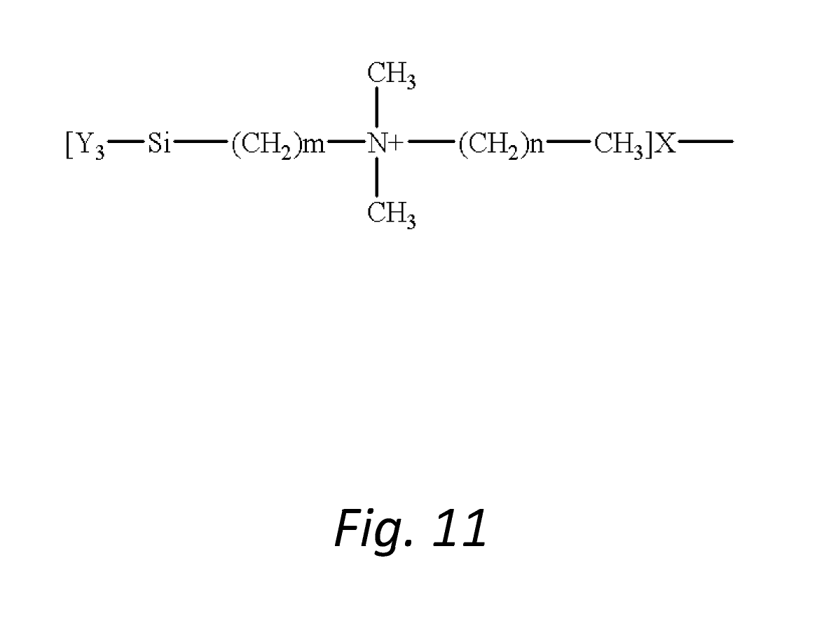

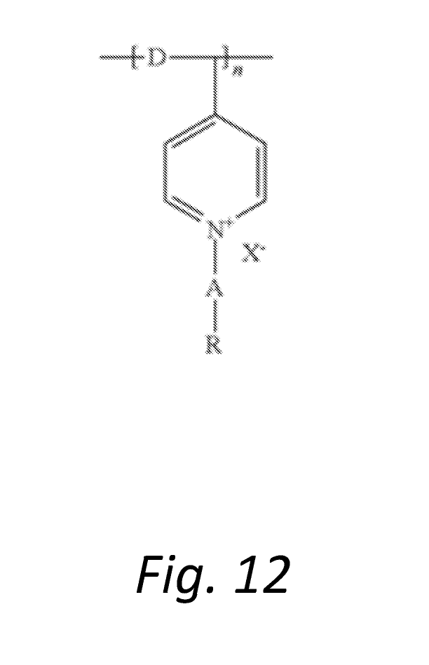

The antimicrobial material must include at least one quarternary ammonium salt (QAS) and/or at least one siliylated polyvinylpyrrolidone (PVP) quarternized salt. FIG. 11 shows the chemical formulation for the QAS. In the formulation shown, m+n is 16 to 19, m is 1 to 6, and n is 13 to 17; or m+n is 20 to 23, m is 4 to 11 and n is 9 to 17; X is a halogen; and Y is a hydrolysable radical or hydroxy group. The formula of a suitable siliylated PVP quarternized salt is illustrated in FIG. 12. In this formulation, R is a substituted or unsubstituted phenyl group; A is a C1-6 alkyl chain; D is a C1-6 alkyl chain; X is a halogen, and n is at least 2

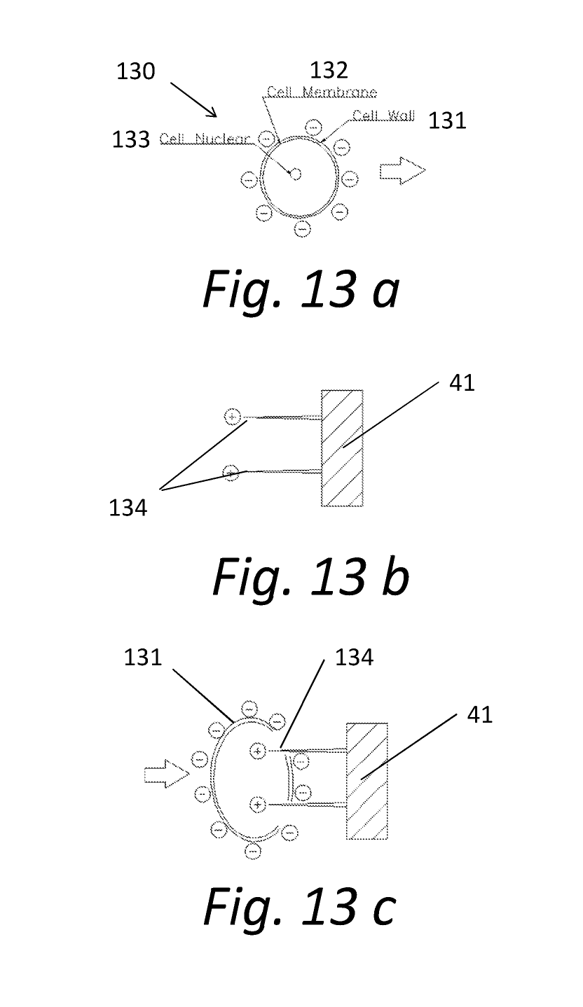

FIGS. 13a, b and c, depict the antimicrobial process utilized herein. FIG. 13a shows a cell 130. The cell 130 includes a cell wall 131, an inner cell membrane 132, and a cell nucleus 133. The cell 130 has a negative charge. FIG. 13b depicts generally the structure of a microstructure 41 on the surface of a disk 12 that has been coated with a suitable antimicrobial material. Nano needles 134 are formed from the salt (QAS or siliylated PVP quarternized). The nano needles 134 are sufficiently rigid to pierce the cell wall 131 and the cell membrane 132. As shown in FIG. 13b, the nano needles are positively charged. This creates an electrical attraction between the cells 130 and the nano needles 134, so that the cells 130 are drawn towards the nano needles 134. As illustrated in FIG. 13c, the electrical attraction of the negatively charged cells 130 to the sharp, rigid, positively charged nano needles 134 brings the cells 130 into contact with the nano needles 134. As the cell 130 contacts the nano needles 134, the nano needles 134 naturally puncture and destroy the cell wall 131 and membrane 132. This not only kills the cell 130 but also releases the internal components of the cell 130 into the carrier fluid.

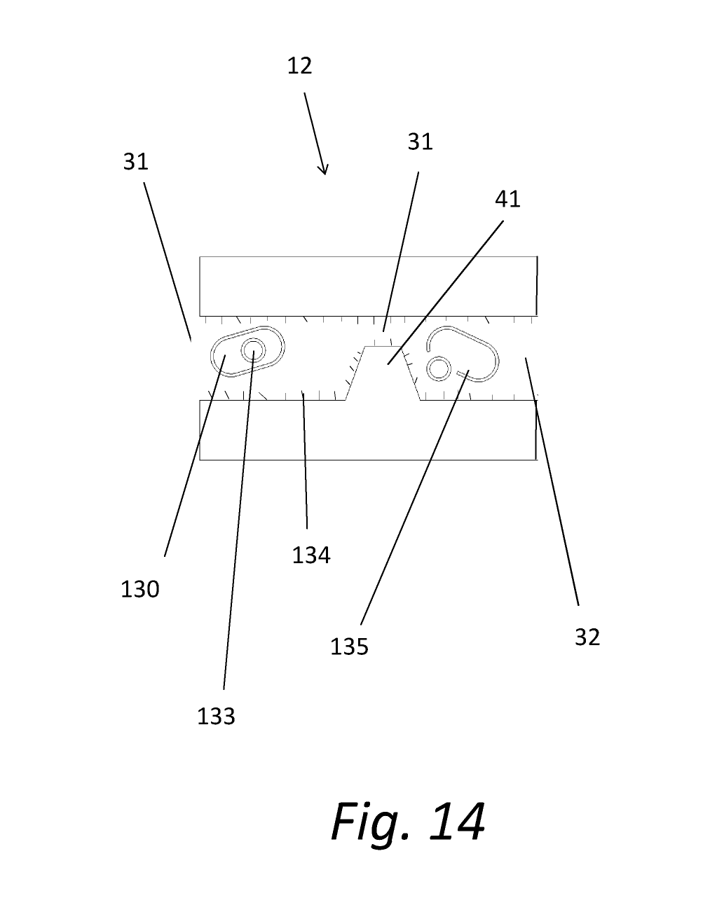

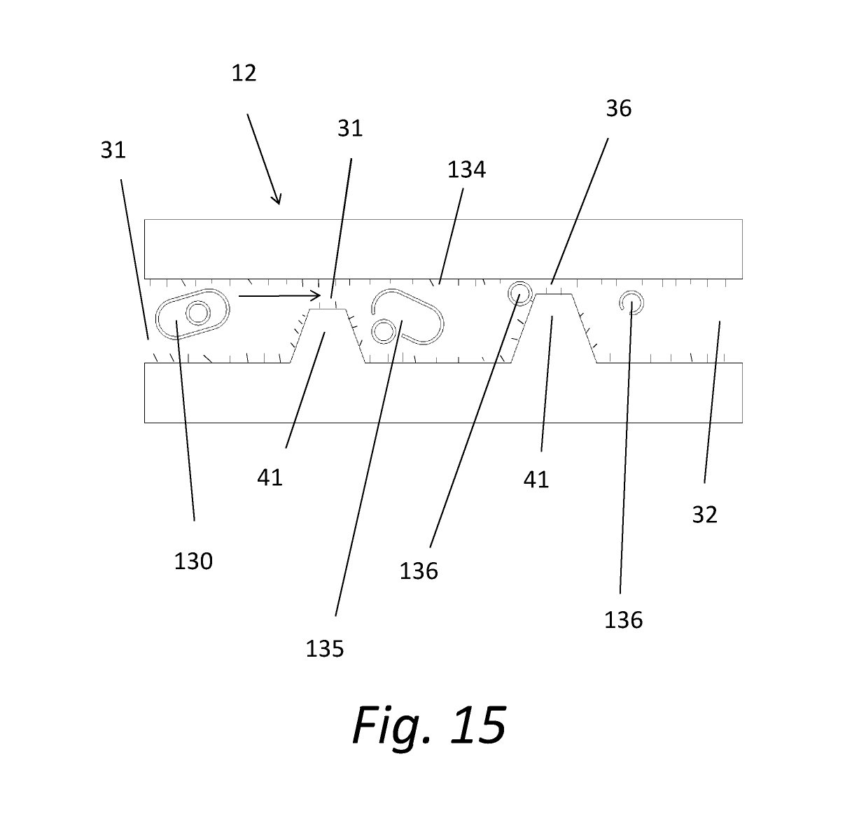

FIGS. 14 and 15 illustrate a fluid flow in a microstructure disk 12. The fluid flow enters an inlet channel 31 carrying cells 130. The cells 130 are electrically attracted to come into contact with the nano needles 134 on the microstructures 41. When the cell 130 contacts a nano needle 134, the cell wall 135 is punctured, and the cell 130 is destroyed. The destroyed cell wall 135 allows the contents of the cell 130 to be released into the carrier fluid. The large plurality of microstructures 41 in the disk assembly 20 ensure that all or nearly all the bacteria cells in the fluid flow are destroyed. As shown in FIG. 15, once the cell wall 131 has been punctured, the contents of the cell, including the cell nucleus 136 are released into the fluid flow. As shown, the cell nucleus 136 may itself be punctured.

Engineering factors for the filtering system 10 may be modified in applications where human health considerations are involved. In such applications, the height of the inlet channels 31 might be smaller than the diameter of the subject cell to ensure that all the cells in the fluid flow are killed. For applications in which the goal is to release the cell contents into the fluid for analysis of the contents of the cells, the height of the inlet channel 31 need not be as small as the cell, thereby allowing a greater flow rate.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel, or may be performed at different times.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. An antibacterial filtering system, comprising:
at least one microstructure disk contained in a housing through which there is a fluid flow containing the cells to be eliminated;
the microstructure disk comprising a plurality of raised microstructures thereon, the microstructures being positioned to form inlet channels and outlet channels for the fluid flow to pass through;
the microstructures being coated with an antimicrobial material that is bonded to the microstructures.

2. The system of claim 1, wherein:
the antimicrobial material includes at least one quarternary ammonium salt (QAS).

3. The system of claim 2, wherein:
the chemical formula for at least one QAS in the antimicrobial material is 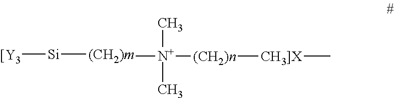
where m+n is 16 to 19, m is 1 to 6, and n is 13 to 17; X is a halogen; and Y is a hydrolysable radical or hydroxy group.

4. The system of claim 2, wherein:
the chemical formula for at least one QAS in the antimicrobial material is 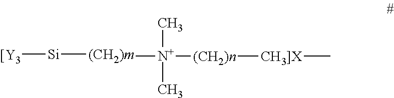
where m+n is 20 to 23, m is 4 to 11 and n is 9 to 17; X is a halogen; and Y is a hydrolysable radical or hydroxy group.

5. The system of claim 2, wherein:
the antimicrobial material includes at least one siliylated polyvinylpyrrolidone (PVP) quarternized salt.

6. The system of claim 2, wherein:
the chemical formula for at least one siliylated PVP quarternized salt in the antimicrobial material is 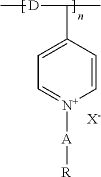
where R is a substituted or unsubstituted phenyl group; A is a C1-6 alkyl chain; D is a C1-6 alkyl chain; X is a halogen; and n is at least 2.

7. The system of claim 1, wherein:
a plurality of nano needles are formed during the application of the antimicrobial material, the nano needles protruding from the surface of the microstructures.

8. The system of claim 7, wherein:
cells included in the fluid flow contact at least one of the nano needles, thereby rupturing at least one of a cell wall and a cell membrane such that the cell is destroyed.

9. The system of claim 1, wherein:
a plurality of microstructure disks forms a microstructure disk assembly that is contained in the housing.

10. The system of claim 9, wherein:
the microstructure disks form an array.

11. The system of claim 10, wherein:
adjacent microstructure disks in the array are spaced apart and physically connected to each other, a boundary of the inlet and outlet channels being formed by a surface of the adjacent microstructure disk.

12. The system of claim 1, wherein:
the microstructures are spaced such that cross channels are formed to create fluid flow between inlet channels and between outlet channels.

13. The system of claim 1, wherein:
the microstructures are sized relative to the size of cells contained in the fluid flow.

14. The system of claim 1, wherein:
the microstructures are sized such that inlet and outlet channels are formed with a cross section smaller than a cross section of cells contained in the fluid flow.

15. The system of claim 1, wherein:
the microstructures are sized such that inlet and outlet channels are formed with a cross section larger than a cross section of cells contained in the fluid flow.
